(19) United States
(12) Patent Application Publication
(10) Pub. No.: US 2012/0088232 A1
(43) Pub. Date: Apr. 12, 2012

(54) Aptamer-Based Device For Detection Of Cancer Markers And Methods Of Use

(75) Inventors: Adam Wanekaya, Springfield, MO (US); Robert DeLong, Springfield, MO (US); Bhaskar Datta, Springfield, MO (US)

(73) Assignee: Missouri State University, Springfield, MO (US)

(21) Appl. No.: 13/250,451

(22) Filed: Sep. 30, 2011

Related U.S. Application Data
(60) Provisional application No. 61/388,389, filed on Sep. 30, 2010.

(51) Int. Cl. G01N 33/53; G01N 21/64; C07H 21/00; G01N 21/49; C12Q 1/68; B82Y 15/00

(52) U.S. Cl. 435/6.1; 436/501; 536/23.1; 422/69; 977/773; 977/774; 977/902

(57) ABSTRACT

Systems and methods are provided for detecting and quantitating one or more compounds or molecules in a sample. An aptamer-based point-of-care device (such as a strip) is described for rapid detection of target molecules such as the cancer marker p-glycoprotein (Pgp). Fluorescent molecules or gold nanoparticles may be used to detect the binding between a target molecule and the aptamer. By way of example, fluorescence resonance energy transfer (FRET) or Dynamic Light Scattering (DLS) may be used for detecting the physical and/or chemical changes caused by the binding of the aptamers to the target molecules.

### RELATED APPLICATIONS

This application claims priority to U.S. Patent application 61/388,389 filed Sep. 30, 2010, the entire content of which is hereby incorporated by reference into this application.

### BACKGROUND

1. Field of the Invention

The disclosure relates to new systems and methods for detecting and quantifying one or more compounds in a sample. More particularly, the disclosure relates to the use of aptamer-based strips for the detection of proteins or other compounds in a patient sample.

2. Description of Related Art

Various biological molecules, such as nucleic acids, proteins, lipids, and other chemicals, play important roles in the structure and function of many life forms. Many different systems have been used to detect the presence of a particular biological molecule in a complex sample. For example, antibodies have been used to detect the presence of a protein in blood samples, and more recently, DNA microarrays have been developed to identify polynucleotides and study gene expression. Most existing systems are designed to detect the presence of a single type or single category of target molecule.

RNA and DNA aptamers bind to a wide variety of target molecules with great affinity and specificity and have been shown to be capable of substituting for antibodies in various applications (Jayasena, “Aptamers: an emerging class of molecules that rival antibodies in diagnostics.” Clin. Chem., 45(9):1628-50, 1999; Morris et al., “High affinity ligands from in vitro selection: complex targets.” Proc. Natl. Acad. Sci., USA, 95(6):2902-7, 1998). The relatively fast selection process of specific aptamers and the inexpensive synthesis of DNA aptamers make them an attractive alternative for antibody-based detection of biological molecules.

In comparison to antibodies, aptamers have numerous advantages as their selection does not depend on animals or cells. In addition, aptamers may be produced by chemical synthesis with high accuracy and reproducibility and reporter molecules such as fluorophores can be attached at precise locations. Although aptamers may be denatured, the process is normally reversible, making them suitable for long term storage and transport at ambient temperature. Aptamers are also relatively more stable at ambient conditions and also under a wide range of buffer conditions. Additionally, nucleic acid probes can also be labeled by radioisotope, biotin, or fluorescent tags and can be used to detect targets under various conditions.

In order to monitor the association between an aptamer and its target more accurately, a signal transduction mechanism need to be devised with a quantifiable read-out. Fluorescent techniques offer excellent choices for signal transduction because of their nondestructive and highly sensitive nature. Several fluorescence techniques have been developed in aptamer assay, including, for example, fluorescence anisotropy, and fluorescence resonance energy transfer (FRET), as well as fluorescence quenching. See e.g., Fang et al., Anal. Chem. 73, 5752-5757 (2001); Li, et al., Biochem. Biophys. Res. Commun. 292, 31-40 (2002); and Nutiu and Li, Chem. Eur. J. 10, 1868-1876 (2004). All these signal-transduction techniques have their individual strength and weakness. For instance, although FRET- or fluorescence-quenching-based probes can quantify target concentrations with changes in fluorescence intensity, both methods are sensitive to the solution environment. More importantly, because of interference from background signals, both fluorescence-based methods have significant limitations in analyzing proteins in their native environments.

When monitoring a protein in its native environment, there are usually two significant background-signal sources. The first one is the probe itself. For example, when a quenching-based FRET molecular probe is used for protein studies, the probe always has some incomplete quenching, resulting in a significant probe background. Moreover, in a native biological environment, there are many potential sources for false positive signals of the molecular probe for protein analysis. The second source of background signal comes from the native fluorescence of the biological environment where the target protein resides. There are many molecular species in a biological environment, some of which will give a strong fluorescence background signal upon excitation. These problems decrease the sensitivity and specificity of currently available assays. Despite extensive research and development in bio-analysis, effective solutions to these problems remain limited.

### SUMMARY OF INVENTION

Research has linked many biological molecules to human disease. Numerous examples have been shown where the overactivity or inactivity of biomolecules are responsible for the pathology underlying chronic and infectious diseases. Examples may include but are not limited to insulin, oncogenes, or tumor suppressors. Antibodies have played a prominent role in the field and have been widely used for recognizing normal and aberrant structures, as probes, in immuno-precipitations, for use in western blotting and enzyme-linked immunosorbance assays and many more commercial applications. Aptamers, which are oligomeric or polymeric nucleic acids, may bind and recognize these biomolecules as well as, if not better than antibodies. More importantly, aptamer mediated technology has distinct advantages over antibodies.

The disclosed instrumentalities advance the art by providing systems and methods for detecting and quantitating proteins or other biological molecules (referred to as “target molecule(s)”) in a sample with improved sensitivity and selectivity. The target molecule may be a protein, a carbohydrate, a lipid, a nucleic acid or any other biomolecule present in cells or tissues. Alternatively, the target molecule may be a chemical, an element, a heavy metal, or other materials of interest. In one aspect, the target molecule is not a polynucleotide.

The sample may contain the target molecule along with other molecules. The sample may be obtained from sources such as a human, an animal, a cell culture, a contaminated material or a material generated in an industrial process. One of the objectives of the instant disclosure is to develop an easy-to-use device for detecting a target molecule in a patient sample at the point-of-care location. Another objective of the present disclosure is to develop a highly sensitive, selective, accurate and rapid platform for the detection of Pgp or other cancer markers using aptamers labeled with appropriate tags.

In one embodiment, a method and a system are disclosed for detecting a target molecule in a sample. The method may be performed by (a) contacting the sample with a polynucleotide molecule capable of binding to said target molecule, wherein said polynucleotide molecule comprises an aptamer, said aptamer being capable of binding to said target molecule; (b) allowing the target molecule to bind to said aptamer; and (c) quantitating the amount of said target molecule bound to said aptamer. In one aspect, the aptamer may be conjugated to a solid support, such as, a nanoparticle, a quantum dot, or other nanomaterials. In another aspect, the aptamer is conjugated to nanoparticles. The quantitating step may use techniques for measuring changes in at least one property of the nanoparticle, said at least one property being selected from the group consisting of size, color, strength of fluorescent signal, wavelength, magnetic property, light scatter property and other spectroscopic changes. In one embodiment, the quantitating step employs Dynamic Light Scattering (DLS) spectroscopy technique. In another aspect, the contacting step may occur on a strip, in bulk or in a solution, and more preferably on a strip.

In another aspect, the aptamer may be chemically modified for enhanced performance either in its backbone, base or sugar moieties and in any location within its sequence.

In one aspect, aptamers that bind specifically to one or more of the target molecules may be designed, synthesized and labeled with certain reporter molecules before loading onto the strip. Examples of reporter molecules may include but are not limited to fluorescent molecules, gold nanoparticles, and so on. Application of the patient sample on the strip may result in the specific binding of the one or more markers that are present in the sample to the aptamers. As the sample flows along the strip, the marker-bound aptamer nanoconjugates may be trapped by capture molecules that have been attached to the strip. The strip may be washed to remove non-specific binding by other proteins. The strip may then be read visually or with an instrument that detects the fluorescent light to quantify the amount of the marker. Examples of such instruments include but are not limited to a hand-held portable fluorescent imager, stationary or mobile fluorescence reader.

In one aspect, the well known lateral flow format may be employed to fabricate the strip. Unlike traditional lateral flow strips used in antibody-based assay, the strip of the instant disclosure preferably combines traditional lateral flow kit design with contemporary fluorescent nanoparticle labels and aptamer assays. Moreover, unlike traditional lateral flow strips that incorporate several membranes, the instant strip preferably uses only one membrane. In one aspect, the membrane is made of a large-pore single layer hydrophilic material that is non-protein binding in nature. This material may fulfill all of the required functionalities of the components of the traditional lateral flow device, namely, as a sample pad, a conjugate pad, a membrane, and an adsorbent pad.

In one embodiment, one single polynucleotide molecule may be used which binds to the target molecule and the capture molecule in a sequential manner. In one aspect, the polynucleotide molecule of the instant disclosure may contain one aptamer, or more preferably, two or more aptamers. In another aspect, the polynucleotide molecule contains a first aptamer and a second aptamer, with the first aptamer and the second aptamer separated by a third sequence (also referred to as the “inter-aptamer segment”). The inter-aptamer segment is capable of binding to a reporter molecule, such as a fluorescent moiety. When the target molecule in the sample binds to the first aptamer, it may alter the conformation of the first aptamer or the conformation of the entire polynucleotide molecule. Such conformational change may make the second aptamer more accessible to its binding partner and thus increasing the binding between the second aptamer and the trapping molecule(s). The trapping molecules may be pre-attached to a strip in order to retain the target molecules. Examples of trapping molecules may include but are not limited to ATP, among others.

In another aspect, the polynucleotide molecule may contain a first aptamer and a second aptamer, with an inter-aptamer segment separating the first and the second aptamers. The first aptamer may have a secondary or tertiary conformation that may be changed upon binding of the first aptamer to the target molecule.

In another aspect, the polynucleotide molecule (also referred to as “the testing molecule”) of the instant disclosure may have attached to it a first reporter moiety and a second reporter moiety. In another aspect, the binding site for the first reporter moiety and the binding site for the second reporter moiety are separated by the first aptamer. When the target molecule in the sample binds to the first aptamer, the distance between these two reporter moieties may change which, in turn may affect the FRET efficiency between the first reporter moiety and the second reporter moiety. The amount of the target molecules bound to the testing molecule may be calculated based on the change in the FRET efficiency. In another aspect, the first reporter moiety and the second reporter moiety are reporters that can work together in FRET. Examples of suitable first and second reporter moieties include but are not limited to fluorescein and Cy3, which is a commonly known fluorescent FRET pair.

In another aspect, the polynucleotide molecule may have attached to its inter-aptamer segment at least one reporter moiety. The binding of the target molecule to the first aptamer may alter the secondary or tertiary structure of the first aptamer which may, in turn, change the nature and/or the intensity of signals emitted by the reporter moiety. In another aspect, the reporter to be used for this aspect of the invention is a reporter moiety whose signals change according to the structural changes of the polynucleotide surrounding the binding site. Example of such reporters include but are not limited to 2-aminopurine, and so on.

In another aspect, the polynucleotide molecule of the instant disclosure may form a hairpin or stem-loop structure when one sequence at the 5′ end of the polynucleotide is complementary to another sequence in the 3′ end of the polynucleotide molecule. The binding of target molecule to the aptamer(s) on the polynucleotide molecule may alter the hairpin or stem-loop structure and thus changing the distance between the different reporter moieties attached to said polynucleotide molecule.

The instant disclosure thus provides a system for detecting and/or quantitating a target molecule in a sample. The system may contain a device and a polynucleotide molecule, with at least one trapping molecule attached to the device. The polynucleotide molecule may contain a first aptamer and a second aptamer, with the first aptamer and the second aptamer being separated by an inter-aptamer segment. The first aptamer has a secondary or tertiary conformation that is capable of being changed to a different secondary or tertiary conformation upon binding of the first aptamer to the target molecule. The at least one trapping molecule is capable of binding to the second aptamer of the polynucleotide molecule. In one aspect, the device is a platform that is capable of holding the sample and allowing the sample to flow freely in a lateral fashion. In another aspect, the device is a strip. In another aspect, the disclosed methods may be practiced using a disposable strip that contains all the testing reagents. The results may be observed by eye or with a reading instrument on site.

According to the present disclosure, in order to detect and/or quantitate a target molecule in a sample, a polynucleotide capable of binding to the target molecule may be caused to be in contact with the sample, preferably on a strip pre-loaded with at least one trapping molecule (also referred to as “capture molecule”). The polynucleotide molecule may contain a first aptamer and a second aptamer, with the first aptamer and the second aptamer being separated by an inter-aptamer segment. In one aspect, the inter-aptamer segment is capable of binding to a reporter, such as a fluorescent moiety. The target molecule is allowed to bind to the first aptamer and the amount of the target molecule bound to the first sequence may be quantitated according to methods described herein or other methods commonly known in the field. The target molecule may be different biological molecules, such as nucleic acids, proteins, lipids, or other chemicals. In one aspect, the target molecule is not a nucleic acid or poly- or oligonucleotide. In another aspect, the target molecule is a protein, or a cancer marker, such as p-glycoprotein (Pgp).

### BRIEF DESCRIPTION OF THE DRAWINGS

FIG. 1 shows the UV-Vis spectrum of gold nanoparticles (GNPs) and aptamer-conjugated GNPs.

FIG. 2 shows the size distribution of GNPs (top panel) and aptamer-conjugated GNPs with 1.41 nM of thrombin (bottom panel).

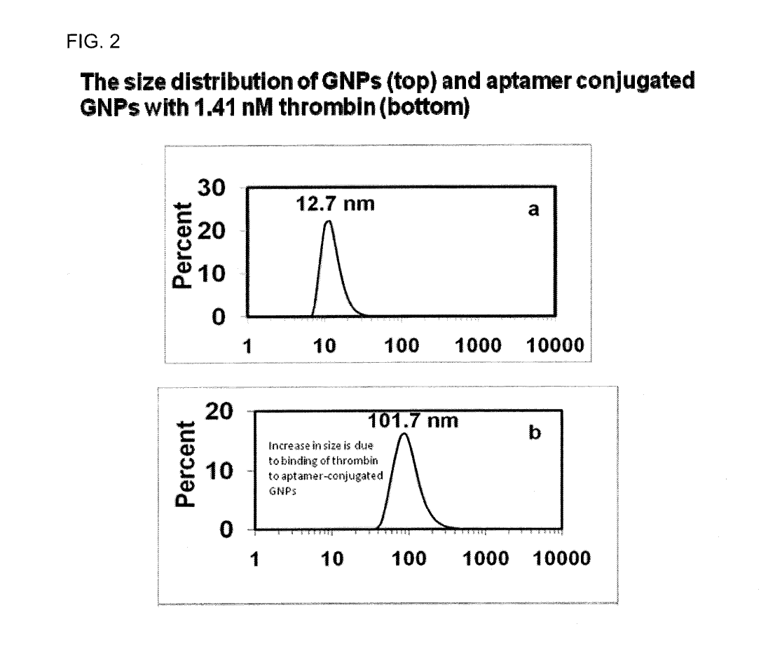

FIG. 3 shows the image of the GNPs under transmission electron microscope (TEM).

FIG. 4 shows the DLS (Dynamic Light Scattering) spectroscopy measurements upon addition of increasing concentration of thrombin to the GNPs.

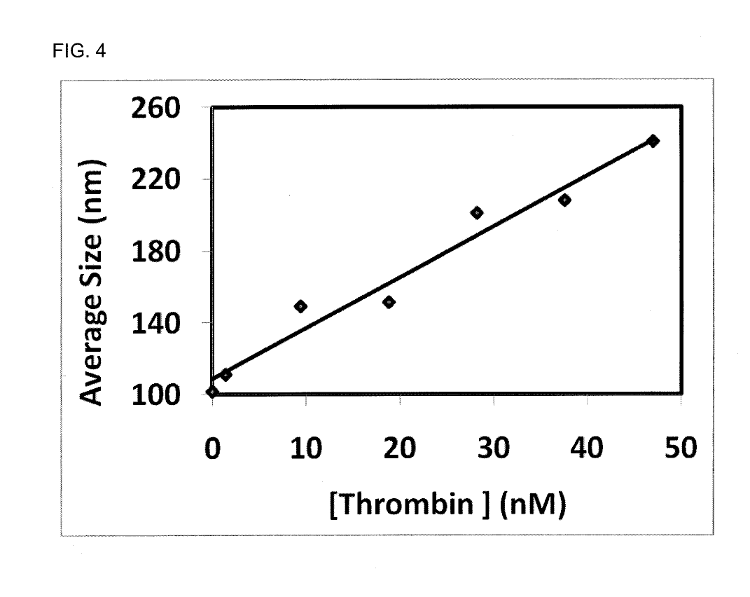

FIG. 5 is a schematic representation showing the functionalization of gold with thiolated thrombin aptamer followed by binding with thrombin (top panel) and DLS analysis depicting corresponding increase in hydrodynamic diameter upon the addition of thrombin (bottom panel).

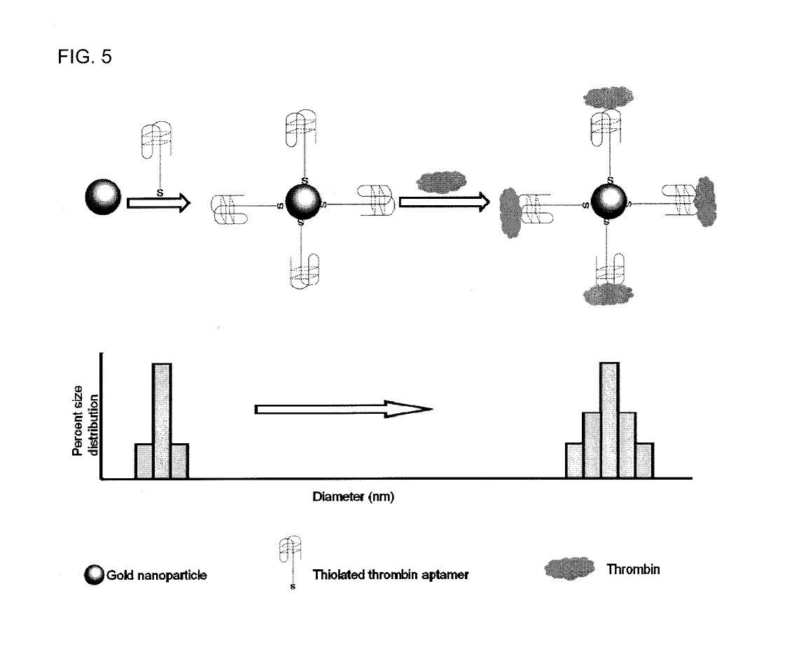

FIG. 6 is a schematic representation showing the structures of Construct A and Construct B, respectively.

FIG. 7 shows the size and distribution of (a) GNPs (12.7 nm) and (b) aptamer-conjugated GNPs (101.7 nm).

FIG. 8 shows the size and distribution of aptamer-conjugated GNPs after addition of thrombin resulting in 28 nM of thrombin in final solution.

FIG. 9 shows the effect of varying the concentration of gold nanoparticles on the hydrodynamic size of the nanoconjugates. [Thrombin]=30 nM, aptamer concentration=1 OD.

FIG. 10 shows the effect of varying the amount of aptamer conjugated to the gold nanoparticles. [Thrombin]=30 nM.

FIG. 11 shows the average size of the Apt-GNP nanoconjugates after addition of thrombin.

FIG. 12 shows the plot of average size vs thrombin concentration.

FIG. 13 shows the average size of the Apt-GNP nanoconjugates after addition of lysozyme.

### DETAILED DESCRIPTION

The instant disclosure provides a point-of-care device for rapid detection of cancer markers and other proteins. Accumulating evidence suggests that prior levels of certain proteins, such as the p-glycoprotein (Pgp), may be an important indicator for predicting how effective certain cancer treatment will be in a patient. Pgp has been implicated in a number of cancers, such as breast cancer, bone cancer, and childhood cranial cancer, among others. Expression of Pgp by tumor cells appears to be associated with an estimated nine-fold increase in the odds of death and a five-fold increase in the odds of metastasis. The same or similar pattern has been found in many different cancer cases. Therefore, accurate detection of Pgp in cancer patients is important for assessing the prognosis of cancer patients. However, current methods for the detection of Pgp are time consuming and requires special attention of very skilled personnel. Moreover, the currently available methods are not very sensitive and specific, which may lead to inaccurate measurement.

The present disclosure provides an aptamer-based point-of-care strip for rapid detection of target molecules such as the cancer marker p-glycoprotein (Pgp). The aptamers may be designed and selected using well-known aptamer selection techniques. The aptamer-based polynucleotide molecules may be used in a solution-based or a solid-based detection system.

In one embodiment, the device disclosed herein uses a single aptamer as the recognition molecule. Aptamers that bind to specific target molecules may be selected by synthesizing an initial heterogeneous population of oligonucleotides, and then selecting oligonucleotides within that population that bind tightly to a particular target molecule. Once an aptamer that binds to a particular target molecule has been identified, it may be produced using a variety of techniques commonly known in the art, for instance, by cloning, by amplification using polymerase chain reaction (PCR), by in intro transcription, among others.

The synthesis of a heterogeneous population of oligonucleotides and the selection of aptamers within that population may be performed using a procedure known as the Systematic Evolution of Ligands by Exponential Enrichment or SELEX. The SELEX method has been described in the literature. See e.g., Gold et al., U.S. Pat. Nos. 5,270,163 and 5,567,588; Fitzwater et al., “A SELEX Primer,” Methods in Enzymology, 267:275-301 (1996); and in Ellington and Szostak, “In Vitro Selection of RNA Molecules that Bind Specific Ligands,” Nature, 346:818-22. In SELEX, random sequence mixtures of nucleic acids are generated and individual molecules isolated from the population by allowing them to bind to or be utilized by an enzyme, receptor or cellular target. The selected species are then amplified and multiple cycles of selection and amplification foster competition between active compounds and eventually result in the purification of those few ligands that have the highest affinity or efficacy for a given target.

The term “aptamer” may be used to refer to oligonucleic acid or peptide molecules that bind to a specific target molecule(s). For purpose of this disclosure, unless otherwise specified, the term “aptamer” refers to an oligonucleotide, which, by itself, is capable of binding a specific biological molecules, or more preferably, to a naturally occurring protein. A “naturally occurring” molecule is a molecule that exist in the body or the cells of a non-transgenic individual regardless of the health status of said individual. The terms “polynucleotide” and “oligonucleotides” are used interchangeably in this disclosure to refer to any nucleic acid molecules having a total of three or more single nucleotides in a continuous string.

The aptamer-based polynucleotide molecules may be bioengineered to undergo conformation changes upon aptamer interaction with a target molecule. The term “conformation” refers to any structural features of a protein or a nucleic acid other than its primary sequence. A conformational change in a polynucleotide is an alteration in the secondary and/or tertiary structure of the polynucleotide. In one aspect, a conformational change may result in the addition and/or deletion of basepairing interactions in and between different segments with the polynucleotide.

Examples of reporter moieties that may be used include but are not limited to dyes, enzymes, or other reagents, or pairs of reagents, that are sensitive to the conformational change or other change in the physical properties of the testing molecules. By way of example, fluorescent molecules or gold nanoparticles may be used to detect the binding between the target molecules and the DNA aptamers. In certain cases, reporter moieties may be incorporated into the aptamer prior to transcription, while in other instances, reporter moieties may be incorporated into the aptamer post-transcriptionally.

In one aspect, the first reporter moiety may be an energy absorbing moiety and the second reporter moiety may be a fluorescence emitting moiety, such that when the first and second reporter moieties are in sufficiently close proximity, energy transfer (FRET) between the moieties may occur, thereby allowing the emitting moiety to emit fluoresce.

In another aspect, the disclosed device may be used for simultaneously detecting the presence of a plurality of different target molecules in a sample. The device may include a solid support; and a plurality of different aptamer-containing polynucleotides bound to the support. The solid support may be a glass surface to which the polynucleotides are covalently bound. In addition, the solid support may be a planar surface, and the polynucleotides may be distributed on the planar surface in a two-dimensional array. Spots of identical polynucleotides may be located at different points in the two-dimensional array.

The aptamer of the instant disclosure may be configured to bind to target molecule(s) selected from the group consisting of a protein, a steroid, an inorganic molecule and an organic molecule. The aptamer-containing polynucleotide may contain DNA, RNA, modified DNA, modified RNA, or a combination thereof.

In another aspect, the systems and methods may be applied for detecting the presence or absence of one or more different target molecules in a sample. The target molecule may be a protein, a steroid, an organic or inorganic molecule, or a nucleic acid. In one embodiment, the target molecule is a protein. A plurality of the aptamer-containing polynucleotides may be caused to be in contact with sample simultaneously or sequentially. The aptamer-containing polynucleotide(s) may be in a liquid, or may be bound to a solid support, such as a particle or a plate.

It is to be noted that, as used in this specification and the claims, the singular forms “a,” “an,” and “the” include plural referents unless the context clearly dictates otherwise. Thus, for example, reference to “a strip” may include reference to a mixture of two or more strips.

The terms “between” and “at least” as used herein are inclusive. For example, a range of “between 5 and 10” means any amount equal to or greater than 5 but equal to or smaller than 10.

Although this disclosure uses protein binding and detection to demonstrate the systems and methods of the current invention, the systems and methods disclosed herein can be modified for the detection of other target molecules by the modification of the aptamers, nanoparticles (e.g., GNPs) and/or reporter moieties (tags).

Various commercially available products may have been described or used in this disclosure. It is to be recognized that these products are cited for purpose of illustration only. Certain physical and/or chemical properties and composition of the products may be modified without departing from the spirit of the present disclosure. One of ordinary skill in the art may appreciate that under certain circumstances, it may be more desirable or more convenient to alter the physical and/or chemical characteristics or composition of one or more of these products in order to achieve the same or similar objectives as taught by this disclosure.

### EXAMPLES

The following examples are provided to illustrate the present invention, but are not intended to be limiting. The reagents, the reporters and instruments are presented as typical components, and various substitutions or modifications may be made in view of the foregoing disclosure by one of skills in the art without departing from the principle and spirit of the present invention.

### Example 1

### Preparation of Strip as the Platform for the Binding of Aptamer-Conjugated Nanoparticles to Marker Proteins

The well known lateral flow format was utilized to fabricate the strip. Unlike traditional lateral flow strips used in antibody-based assay, this strip combined traditional lateral flow kit design with contemporary fluorescent nanoparticle labels and aptamer assays. Moreover, unlike traditional lateral flow strips that incorporate several membranes, the instant strip used only one membrane that was made of a large-pore single layer hydrophilic material that was non-protein binding in nature. This material fulfilled all of the required functionalities of the components of the traditional lateral flow device, namely, as a sample pad, a conjugate pad, a membrane, and an adsorbent pad. Because the material used to build the membrane did not bind to proteins, a different strategy called “laying down boulders in the stream” was used to construct the test and control lines. Briefly, large micron sized beads were used to conjugate specific proteins and were dispensed and immobilized on the strip where they formed the test and control line. When the test sample and protein conjugates flowed past the “boulders,” binding and signal formation occurred at those locations.

### Example 2

### Binding of Aptamer-Conjugated Nanoparticles to Marker Proteins

Dithiothreitol (DTT), thrombin from bovine plasma, and bovine serum albumin (BSA) were purchased from Sigma Aldrich (St Louis Mo.). Lysozyme was purchased from Sigma-Aldrich (St. Louis, Mo.). Thiolated thrombin with sequence 5′-SH-(CH₂)₆-TTTTTTTTTTGGTTGGTGTGGTTGG-3′ and thiolated lysozyme aptamer, 5′-/5thioMC6-D/ATC TAC GAA TTC ATC AGG GCT AAA GAG TGC AGA GTT ACT TAG-3′ were purchased from Integrated DNA Technologies (Coralville, Iowa). Hydrogen tetrachloroaurate (III) trihydrate (HAuCl₄.3H₂O) was obtained from Fisher Scientific (Fair Lawn, N.J.). Sodium citrate was obtained from Spectrum Chemical Corp (New Brunswick, N.J.). Gel filtration columns (NAP-5) was purchased from GE Healthcare Bio-Sciences Corp (Piscataway, N.J.). DLS Spectroscopy was performed on a Malvern Nanozetasizer (ZS90). The DLS spectrometer was operated at 25° C. with the detector angle at 90°, incident laser wavelength of 633 nm and 4 mW laser power. UV/Vis spectroscopy was conducted on a Perkin Elmer Lamda 650 UV/VIS spectrometer.

GNPs were prepared by the citrate reduction of HAuCl₄.3H₂O according to a modified literature method. See M. A. Hayat, Colloidal gold: principles methods and applications. Academic Press, 1989. Briefly, an aqueous solution of HAuCl₄.3H₂O (1 mM, 500 mL) was brought to reflux while stirring. 50 mL of 38.8 mM trisodium citrate solution was then added rapidly. After 15 minutes, the reaction was stopped and the mixture allowed to cool to room temperature and subsequently filtered through a 0.45 micron filter. The concentration of the GNPs was determined by their absorbance spectra (λ=523 nm) and using appropriate extinction coefficient and was found to be 0.063 μM.

Upon synthesis of the citrate-stabilized GNPs, DLS spectroscopy measurements and TEM images were taken to ascertain their size before conjugation was attempted. All DLS spectroscopy measurements were allowed a two minute equilibration time and were performed in triplicate. The GNPs were then conjugated to the thiolated aptamer using a modified literature protocol that utilized thiol-Au affinity. See H. Xu, X. Mao, Q. Zeng, S. Wang, A.-N. Kawde, and G. Liu, Analytical Chemistry, 2008, 81, 669. The thiol modified oligonucleotides were purchased in a disulfide form which had to be cleaved using DTT. 200 μL of 0.55 OD thiol modified oligonucleotide was mixed with 2 μL of triethylammonium hydroxide (10%) and 7.7 mg DTT. The solution was allowed to react for one hour, and DTT was then removed via extraction with 400 μL ethyl acetate. The extraction was repeated four times to ensure complete removal of DTT. Then, 1 mL of the previously prepared gold nanoparticle solution was added, and the mixture was allowed to set for 24 hours. After this waiting period, the conjugate was slowly aged with addition of PBS until a final concentration of 0.01 M was reached. Following this, the mixture was left at 5° C. for 24 hours. The conjugated were then centrifuged with a Daigger centrifuge at 6000 rpm for 40 minutes. They were washed and recentrifuged, and the supernatant was discarded. The precipitate was redispersed in a 10 mL solution containing 0.0076 g Na₃PO₄.12H₂O, 0.10 g sucrose, and 2.5 μL Tween 20. This was then stored at 5° C. until subsequent experiments were performed.

Gold Nanoparticle Synthesis and Modification

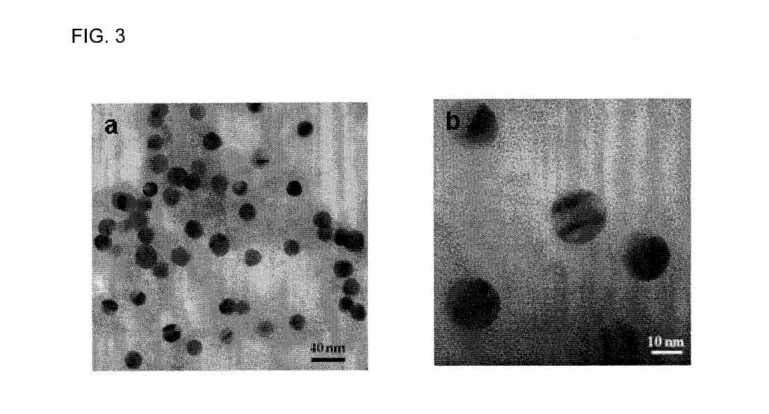

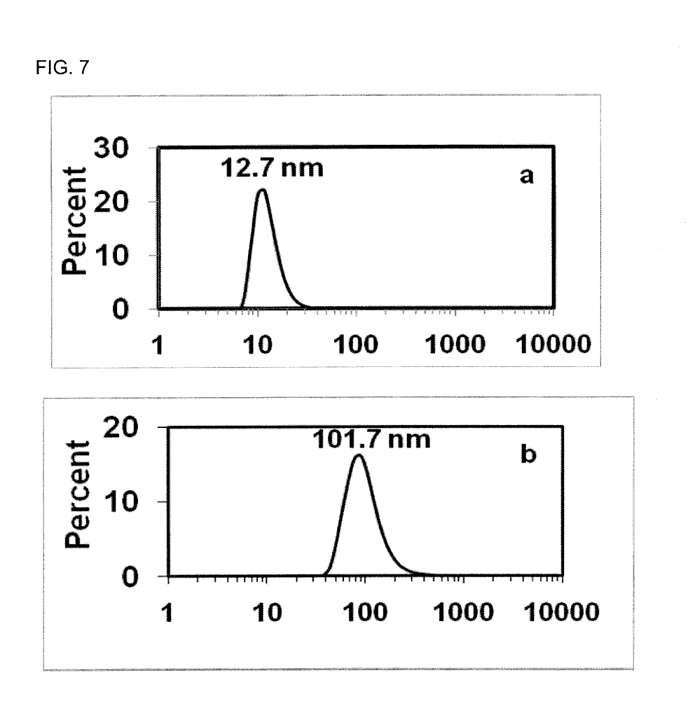

FIG. 3a shows a low magnification TEM image of the citrate stabilized GNPs. The GNPs were well dispersed with a very narrow size distribution. FIG. 3b shows a higher magnification TEM image of the same particles. It is evident that the shape of the nanoparticles was consistently spherical with an average diameter of about 13 nm. DLS spectra were recorded to ascertain the size of these. As can be seen from FIG. 7a, the average size of the GNPs was found to be 12.7 nm, which was consistent with the TEM results above.

Thiolated aptamers were then bound to the GNPs. The conjugation was based on the well known thiol-gold affinity. Further DLS spectroscopy experiments were performed after conjugation, and the average size of the aptamer-conjugated GNPs was found to be 101.7 nm (FIG. 7b). This dramatic diameter change is due to the binding of the thiolated aptamer onto the surface of the gold nanoparticles, as well as the formation of dimers, trimers, and oligomers as the aptamer-gold nanoparticle conjugates formed aggregates. The conjugated nanoparticles remained very stable in solution as confirmed by the DLS data. Even with repeats of triplicate runs all size distributions were very similar.

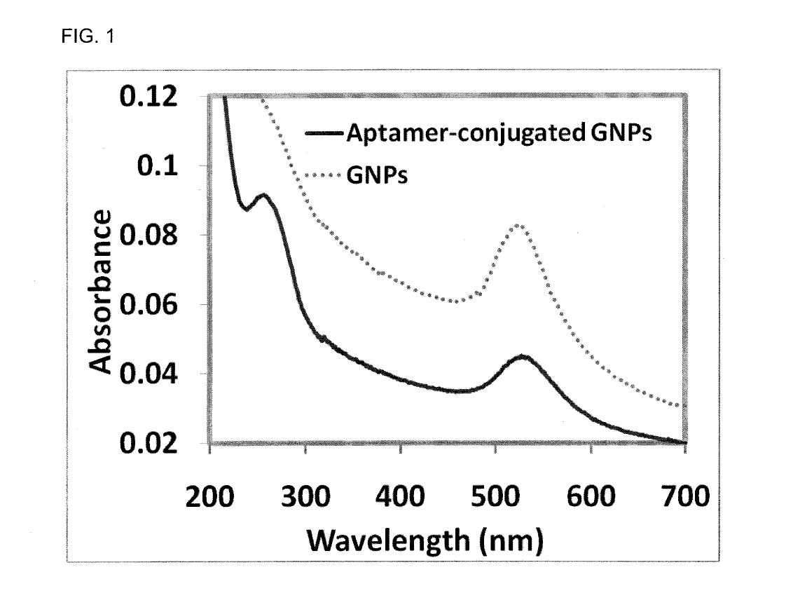

FIG. 1 shows the ultraviolet-visible (UV-VIS) spectra of the GNPs and aptamer-conjugated GNPs. The surface plasmon resonance absorption of the GNPs is clearly evident at 523 nm which undergoes a slight red shift to 530 nm on conjugation of the aptamers. The intensity of the aptamer-conjugated GNPs peak at 530 nm is also reduced compared with the free GNP peak at 523 nm. The red shift and decrease in intensity well known and documented and has been attributed to the decrease in interparticle distance as a result of the binding of the thiolated aptamer onto the GNP surface. See e.g., J. J. Storhoff, A. A. Lazarides, R. C. Mucic, C. A. Mirkin, R. L. Letsinger, and G. C. Schatz, Journal of the American Chemical Society, 2000, 122, 4640. The aptamer conjugation is further confirmed by a weak peak at 260 nm that corresponds to the absorption from the nucleic acid bases on the aptamer. The DLS and UV-VIS spectra demonstrated successful conjugation of the aptamer to the GNPs.

Thrombin Detection and Assay Optimization

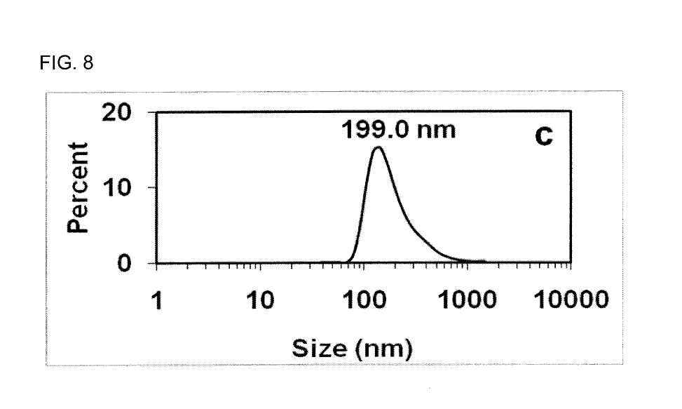

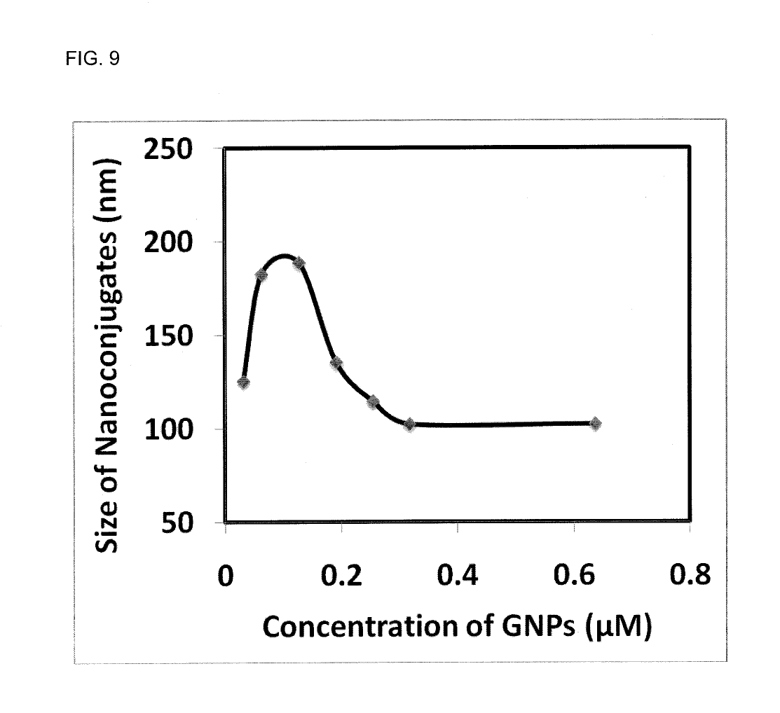

Thrombin was added to aptamer-conjugated GNPs and DLS spectra were recorded upon each addition. Since thrombin has binding sites to which the aptamer can bind to, the thrombin-induced aggregation of the aptamer-conjugated GNPs was expected. These results are outlined in FIG. 8 shows a dramatic increase in size upon addition of thrombin to the aptamer-conjugated GNPs. In order to find ideal conditions for this assay, optimization experiments were performed. The concentration of GNPs and the amount of aptamers conjugated to these GNPs are more likely to affect the analytical signal. The aptamer conjugates of varying optimal conditions were combined with 30 nM of thrombin under the varying conditions outlined below. Variable concentrations of GNPs from 0.0318 to 0.636 μM were prepared. The synthesis of the aptamer-conjugated nanoparticles was carried out as described in the experimental section. It was found that the maximum size distribution shift occurred at about 0.127 μM (FIG. 9). It can be seen that the basic shape of the size distribution curve is an inverted parabola, with the vertex being close to the optimal concentration. Both above and below this point, either too little or too many gold nanoparticles are present to allow optimal conjugation of the aptamer to the gold nanoparticles. Before the maximum the assay has limited amount of gold nanoparticles, in which case, not all aptamers are conjugate, and are thus washed away in the centrifugation step of the synthesis. Conversely, if too many nanoparticles are present, the aptamer may not have room to unfold and achieve enough space to allow successful conjugation to the gold nanoparticles, and would therefore be washed away in the centrifugation step of the synthesis. Even though 0.127 μM was determined to be the optimal concentration for the gold nanoparticles in the synthesis, it is important to note that this size distribution shift is not significantly larger than the original stock gold nanoparticle concentration. For this reason, the original concentration of 0.636 μM was used instead of the slightly larger shift observed for the at 0.127 μM gold nanoparticles.

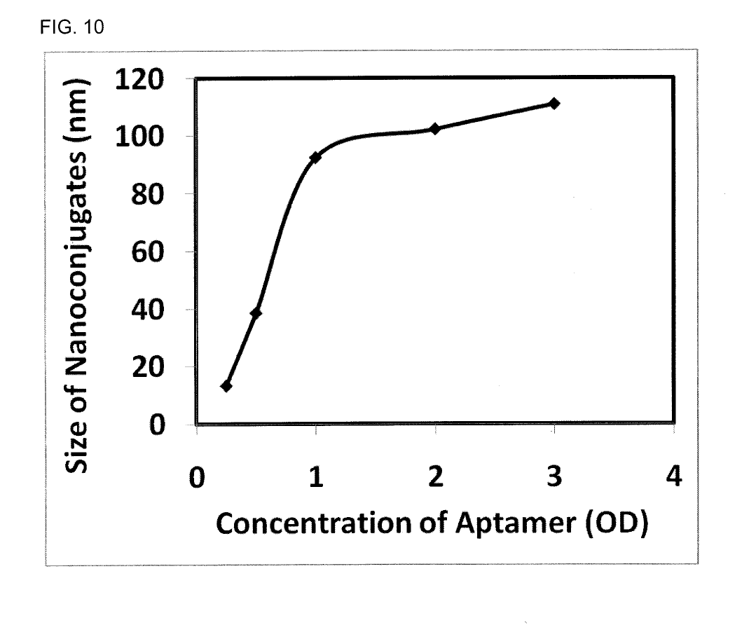

The amount of aptamer conjugated to the GNPs was also optimized. The concentration of the aptamer was varied from 0.25 OD to 3 OD to find the optimum value. Optical densities (ODs) of the aptamers were determined using UV-VIS methods. It was found that size distribution tended upward until around 1OD (FIG. 10). Below this point, significantly less conjugation occurs, and therefore, a smaller size distribution is observed for a fixed amount of added thrombin. Above this point, no more significant conjugation occurs because the solution becomes saturated with the aptamer. Therefore, all the excess aptamer is removed at concentrations above 1OD upon centrifugation of the solution in the synthesis. Again, after 1OD, size changes leveled off, and no significant increase in size distribution was observed. For this reason, 1OD aptamers were used for all further experimentation.

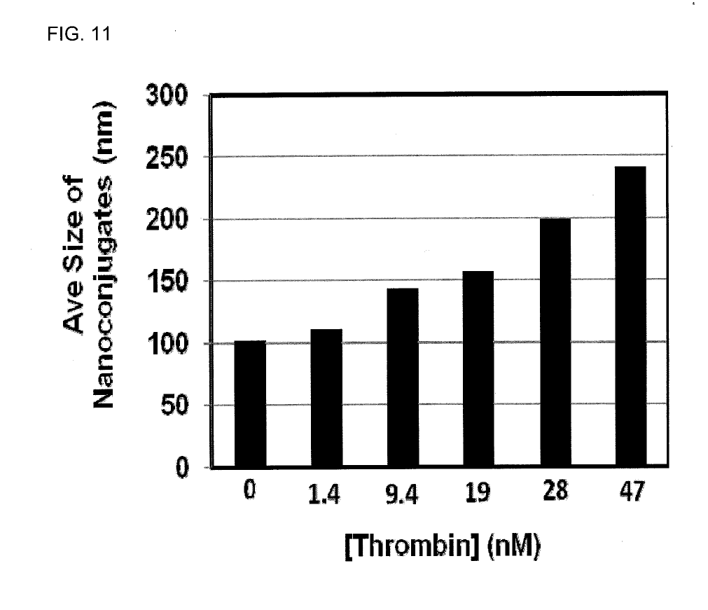

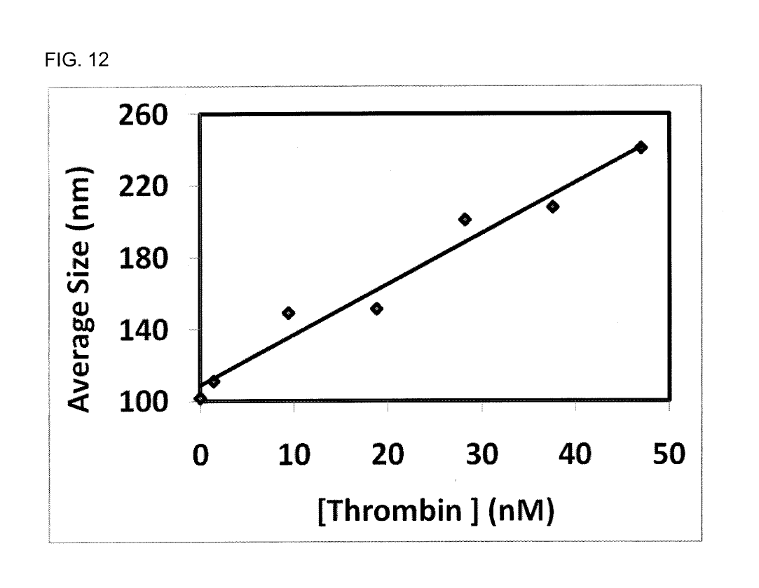

Once the assay was optimized, it was challenged with increasing concentrations of thrombin. As expected, there was a corresponding increase in the hydrodynamic size of the aggregates as shown in FIG. 11. As the concentration of thrombin in the parent solution was increased, there was more aggregation leading to larger hydrodynamic sizes as recorded by DLS spectroscopy. The increase in size was even observed for solutions containing as low as 1.41 nM of thrombin suggesting that our assay was sensitive to low nanomolar concentrations of thrombin. The increase in the hydrodynamic size of the aggregates was found to correspond with the increase in the thrombin concentration (FIG. 12). The detection limit for thrombin was calculated to be 0.2 nM. Our assay was applicable to thrombin concentrations all the way up to 100 nM, where we continued to see increasing shifts in the size distribution of the nanoconjugates.

To examine the selectivity of the assay, we conducted several experiments by challenging the aptamer-conjugated GNPs with bovine serum albumin (BSA). Under the exact same conditions there was no significant increase in size of the particles on adding equivalent concentrations of BSA. Typically, some slight increase in hydrodynamic diameter is not entirely unexpected even on addition of non-specific moieties. However, these were not observed in the experiments as described above.

### Lvsozyme Detection

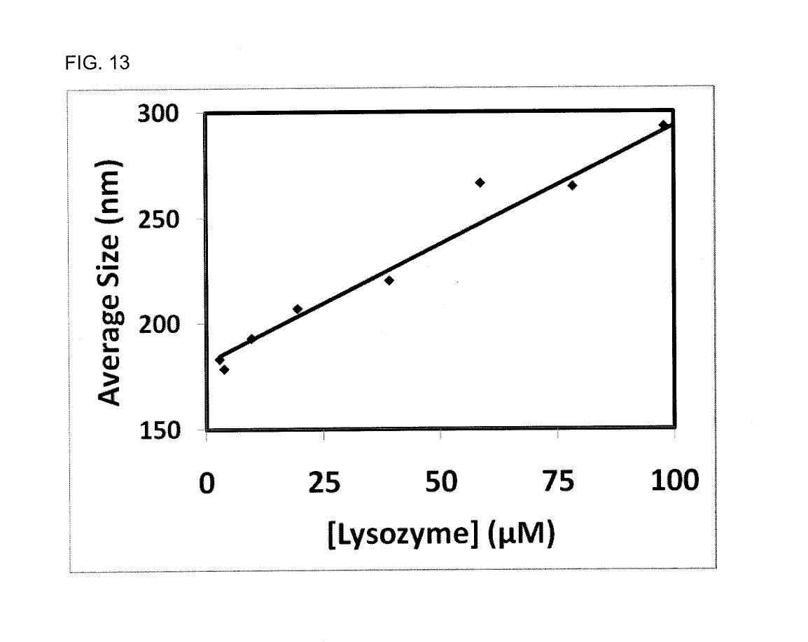

Since the above assay has been shown to work for thrombin, it was extended to another protein, lysozyme, to determine its applicability in a different analyte system. Reduced lysozyme levels have been associated with chronic lung disease in inborns. See M. E. Revenis and M. A. Kaliner, The Journal of Pediatrics, 1992, 121, 262. Thiolated lysozyme-specific aptamer sequences were conjugated to GNPs as described above. Lysozyme was then added to aptamer-conjugated GNPs and DLS spectra were recorded upon each addition. As in the case of thrombin, addition of lysozyme induced aggregation of the aptamer-conjugated GNPs due to the specific binding of the protein to the aptamers. Similarly, the increase in the size of the nanoconjugates was proportional to the concentration of lysozyme. A linear relationship between the hydrodynamic sizes of the nanoconjugates vs the concentration of the lysozyme was demonstrated up to 100 μM (FIG. 13).

These results suggest that nonspecific adsorption of the protein on aptamer-conjugated GNPs is largely undetectable, thus demonstrating the ability of the disclosed assay to discriminate between two different proteins. Numerous proteins including cancer and other disease markers may be analyzed in a timely manner using this type of assay as long as their respective aptamers are conjugated to the GNPs or some other appropriate material. Overall, these results support a novel and highly specific method to rapidly detect proteins by combining DLS spectroscopy and aptamer-conjugated GNPs.

In summary, an extremely facile, rapid specific and selective method has been shown for detecting proteins using aptamer-conjugated GNPs coupled with dynamic light scattering at ambient conditions. The linear increase in the hydrodynamic diameter of aptamer-conjugated GNPs as a result of forming dimers, oligomers or aggregates upon addition of thrombin formed the analytical basis of the assay. A linear dynamic range of up to 100 nM was realized using thrombin as the model analyte enabling the direct detection of as low as 1.41 nM of thrombin. The presence of other interfering proteins such as BSA showed no effect on the assay response. The assay was also successfully demonstrated for lysozyme. Additional studies are underway in our laboratory to better understand the kinetics of binding to aptamer-conjugated GNPs and to test the assay on real samples. While the utility of the assay was demonstrated for protein binding/detection, the assay could easily be designed for the detection of other targets by the modification of GNPs with appropriate aptamers. Therefore, the technology may have positive impact and broad analytical applications in clinical, biomedical and other sectors.

### Example 3

### Aptamer Constructs for Selective Target Detection

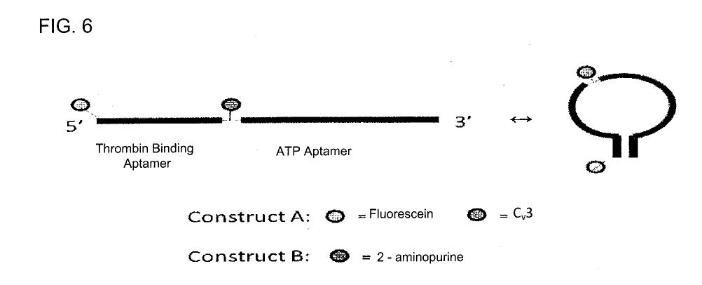

In order to detect proteins in a sample using a lateral strip, an oligonucleotide construct was designed and synthesized which incorporated two known aptamer sequences that bind thrombin and ATP, respectively. As shown in the FIG. 6, the oligonucleotide had both a thrombin binding region and an ATP binding region, and these two binding regions were separated by an inter-aptamer region of several nucleotides in length. In addition, the sequences at the 5′ and 3′ end of the oligonucleotide construct were complementary to each other such that the oligonucleotide would fold into a hairpin structure as shown in FIG. 6.

In construction A, two reporters, fluorescein and Cy3, were conjugated to the 5′ terminus and to the inter-aptamer region, respectively. Fluorescein and Cy3 were known to be a fluorescent FRET pair. Changes in FRET were monitored after separate or sequential addition of thrombin and ATP targets to construct A. FRET efficiencies for experiments on construct A were calculated following standard literature protocols, and the errors are indicative of the standard deviations of triplicate sets of data.

In construct B, the fluorescent base 2-aminopurine was placed in the spacer region between the two aptamer sequences. 2-aminopurine had been used as a reporter molecule for changes in helical conformation of oligonucleotides. Changes in the fluorescence of 2-aminopurine were calculated upon separate and sequential addition of thrombin and ATP.

While constructs A and B were capable of forming a hairpin structure as shown in FIG. 6, the thrombin and ATP aptamers could also fold into other well-known secondary structures. In the presence of thrombin, a higher FRET efficiency was observed as compared to the annealed construct alone, indicating a change in conformation of the oligonucleotide (FRET values (i) and (ii) in Table 1). By contrast, the addition of ATP led to a much smaller increase in FRET efficiency. FRET efficiencies were indicative of the distance between the donor (such as fluorescein) and the acceptor (such as Cy3) fluorophores. Thus, the smaller improvement in FRET upon addition of ATP compared to thrombin suggests that thrombin is more capable of disrupting the oligonucleotide conformation.

TABLE 1
Construct A

| Experiment | FRET Efficiency |
|---|---|
| Annealed construct | 0.75 ± 0.02 |
| + Thrombin | 0.85 ± 0.04⁽ⁱ⁾ |
| + ATP | 0.79 ± 0.01⁽ⁱⁱ⁾ |
| + Thrombin & then ATP | 0.84 ± 0.01 |

The above result was further confirmed by a larger change in the fluorescence of a 2-aminopurine residue in construct B upon addition of thrombin as compared to that of ATP. Comparing the fluorescence of 2-aminopurine in the presence of thrombin and ATP, the values (i) and (ii) in Table 2 showed that thrombin was more capable of disrupting the local structure around the 2-aminopurine than ATP.

TABLE 2
Construct B

| | Fluorescence of |
|---|---|
| Experiment | 2-aminopurine |
| Annealed construct | 1.0 (normalized) |
| + Thrombin | 0.86⁽ⁱ⁾ |
| + ATP | 0.97⁽ⁱⁱ⁾ |
| + Thrombin & then ATP | 0.88 |

The oligonucleotide construct could be modified to contain a first aptamer that binds to other markers of interest. Such markers of interest can be a cancer marker or a disease marker. The marker of interest can be a protein, an organic or inorganic molecule that binds to the first aptamer. One example of such marker is Pgp. The structure of the oligonucleotide could be selectively disrupted by the binding of Pgp which rendered a second aptamer site more accessible. The second aptamer could then be used to latch on to a corresponding molecule (such as ATP) immobilized on a lateral flow strip.

### REFERENCES

All references listed below and those publications, patents, patent applications cited throughout this disclosure are hereby incorporated expressly into this disclosure as if fully reproduced herein.
1 B. Du, Li, Z-P., Liu, C-H., Angewandte Chemie International Edition, 2006, 45, 8022.
2 C. A. Mirkin, Letsinger, R. L., Mucic, R. C., Storhoff, J. J., Nature, 1996, 382, 607.
3 Q. Dai, X. Liu, J. Coutts, L. Austin, and Q. Huo, Journal of the American Chemical Society, 2008, 130, 8138.
4 X. Liu, Q. Dai, L. Austin, J. Coutts, G. Knowles, J. Zou, H. Chen, and Q. Huo, Journal of the American Chemical Society, 2008, 130, 2780.
5 Y. Sun, Xia, Y., Science, 2002, 298, 2176.
6 B. Du, Z. Li, and Y. Cheng, Talanta, 2008, 75, 959.
7 B. Nikoobakht, El-Sayed, M., J. Am. Chem. Soc., 2003, 15, 1957.
8 L. R. Hirsch, J. B. Jackson, A. Lee, N. J. Halas, and J. L. West, Analytical Chemistry, 2003, 75, 2377.
9 C.-C. Huang, Y.-F. Huang, Z. Cao, W. Tan, and H.-T Chang, Analytical Chemistry, 2005, 77, 5735.
10 F. Li, Zhang, J., Cao, X., Wang, L. Li, D., Song, S., Ye, B., Fan, C., Analyst, 2009, DOI: 10.1039/b900900k.
11 J. Liu, Lu, Y., Angewandte Chemie International Edition, 2006, 45, 90.
12 H. Xu, X. Mao, Q. Zeng, S. Wang, A.-N. Kawde, and G. Liu, Analytical Chemistry, 2008, 81, 669.
13 K. Y. Wang, S. McCurdy, R. G. Shea, S. Swaminathan, and P. H. Bolton, Biochemistry, 1993, 32, 1899.
14 V. Bagalkot, Farokhzad, O. C., Langer, R., Jon, S., Angewandte Chemie International Edition, 2006, 45, 8149.
15 K. Maehashi, T. Katsura, K. Kerman, Y. Takamura, K. Matsumoto, and E. Tamiya, Analytical Chemistry, 2007, 79, 782.
16 H. Pandana, Aschenbach, K. H., Gomez, R. D., IEEE Sensors Journal, 2008, 8, 661.
17 C. Washington, ‘Particle Size Analysis in Pharmaceutics and other industries: Theory and Practise’, Ellis Horwood Limited, 1992.
18 L. C. Bock, Griffin, L. C., Latham, J. A., Vermaas E. H., Toole, Nature, 1992, 355, 564.
19 D. M. Tasset, Kubik, M. F., Steiner, W., J. Mol. Biol., 1997, 272, 688.
20 M. E. Revenis and M. A. Kaliner, The Journal of Pediatrics, 1992, 121, 262.
21 M. A. Hayat, ‘Colloidal gold: principles methods and applications.’ Academic Press, 1989.
22 J. J. Storhoff, A. A. Lazarides, R. C. Mucic, C. A. Mirkin, R. L. Letsinger, and G. C. Schatz, Journal of the American Chemical Society, 2000, 122, 4640.

We claim:

1. A method for detecting a target molecule in a sample, said method comprising the steps of:
(a) contacting said sample with a polynucleotide molecule capable of binding to said target molecule, wherein said polynucleotide molecule comprises an aptamer, said aptamer being capable of binding to said target molecule;
(b) allowing said target molecule to bind to said aptamer; and
(c) quantitating the amount of said target molecule bound to said aptamer.

2. The method of claim 1, wherein the quantitating step comprises measuring the amount using Dynamic Light Scattering (DLS) technique.

3. The method of claim 1, wherein the aptamer is conjugated to a solid support.

4. The method of claim 1, wherein the solid support is a nanoparticle, a quantum dot, or other nanomaterials.

5. The method of claim 4, wherein the solid support is a nanoparticle.

6. The method of claim 1, wherein the aptamer is chemically modified for enhanced performance either in its backbone, base or sugar moieties and in any location within its sequence.

7. The method of claim 1, wherein the target molecule is a protein, carbohydrate, lipid or nucleic acid or any other biomolecule present in cells and tissues.

8. The method of claim 1, wherein the target molecule is a chemical, an element, a heavy metal, or other materials of interest.

9. The method of claim 1, wherein the sample is obtained from sources selected from the group consisting of a human, an animal, a cell culture, a contaminated material and a material generated in an industrial process.

10. The method of claim 1, wherein the contacting step occurs on a strip.

11. The method of claim 1, wherein the contacting step occurs in bulk or in a solution.

12. The method of claim 5, wherein the quantitating step comprises measuring the change in at least one property of the nanoparticle, said at least one property being selected from the group consisting of size, color, strength of fluorescent signal, wavelength, magnetic property, light scatter property and other spectroscopic changes.

13. A method for detecting a target molecule in a sample, said method comprising the steps of:
(a) contacting said sample with a polynucleotide molecule capable of binding to said target molecule, wherein said polynucleotide molecule comprises a first aptamer and a second aptamer, said first aptamer and said second aptamer being separated by an inter-aptamer segment, said inter-aptamer segment being capable of binding to a reporter, said sample comprising said target molecule;
(b) allowing said target molecule to bind to said first aptamer; and
(c) quantitating the amount of said target molecule bound to said first aptamer.

14. The method of claim 13, wherein said polynucleotide molecule is housed in an apparatus.

15. The method of claim 13, wherein the target molecule is not a polynucleotide.

16. The method of claim 13, wherein the polynucleotide is capable of forming a hairpin structure.

17. A polynucleotide molecule for detecting a target molecule in a sample, said polynucleotide molecule comprising a first aptamer and a second aptamer, said first aptamer and said second aptamer being separated by an inter-aptamer segment, wherein the first aptamer has a secondary or tertiary conformation, said secondary or tertiary conformation of said first aptamer being capable of changing to a different secondary or tertiary conformation upon binding of said first aptamer to said target molecule.

18. The polynucleotide molecule of claim 17 having attached to it a first reporter moiety and a second reporter moiety, wherein the binding site on said polynucleotide molecule for said first reporter moiety and the binding site on said polynucleotide molecule for said second reporter moiety are separated by the first aptamer, and the binding of said first aptamer to the target molecule alters the FRET efficiency between said first reporter moiety and said second reporter moiety.

19. The polynucleotide molecule of claim 17 having attached to the inter-aptamer segment a reporter moiety, wherein the binding of said first aptamer to the target molecule alters the signal emitted by said reporter moiety.

20. A system for detecting a target molecule in a sample, said system comprising a device and a polynucleotide molecule, said device having attached to it a trapping molecule, wherein the polynucleotide molecule comprises a first aptamer and a second aptamer, said first aptamer and said second aptamer being separated by an inter-aptamer segment, wherein the first aptamer has a secondary or tertiary conformation, said secondary or tertiary conformation of said first aptamer being capable of changing to a different secondary or tertiary conformation upon binding of said first aptamer to the target molecule, said trapping molecule being capable of binding to the second aptamer of said polynucleotide molecule.

21. The system of claim 20, wherein said polynucleotide molecule has attached to it a first reporter moiety and a second reporter moiety, wherein the binding site on said polynucleotide molecule for said first reporter moiety and the binding site on said polynucleotide molecule for said second reporter moiety are separated by the first aptamer, and the binding of said first aptamer to the target molecule alters the FRET efficiency between said first reporter moiety and said second reporter moiety.

22. The system of claim 20, wherein said polynucleotide molecule has attached to the inter-aptamer segment a reporter moiety, wherein the binding of said first aptamer to the target molecule alters the signal emitted by said reporter moiety.

23. The system of claim 20, wherein said device is a strip.
